(19) United States
(12) Patent Application Publication
(10) Pub. No.: US 2014/0228843 A1
(43) Pub. Date: Aug. 14, 2014

(54) SYSTEM AND METHOD FOR RESECTING A VALVE

(71) Applicants: Michael J. O'DONNELL, Ann Arbor, MI (US); Myra EPP, Santa Clara, CA (US); Christine E. STEWART, Minneapolis, MN (US); David Shicheng LI, Ann Arbor, MI (US); Brian D. MATTY, New York, NY (US)

(72) Inventors: Michael J. O'DONNELL, Ann Arbor, MI (US); Myra EPP, Santa Clara, CA (US); Christine E. STEWART, Minneapolis, MN (US); David Shicheng LI, Ann Arbor, MI (US); Brian D. MATTY, New York, NY (US)

(21) Appl. No.: 14/259,006

(22) Filed: Apr. 22, 2014

Related U.S. Application Data
(63) Continuation-in-part of application No. 13/928,312, filed on Jun. 26, 2013, now abandoned.
Application No. 13/928,312 is a continuation of application No. 12/755,361, filed on Apr. 6, 2010, now Pat. No. 8,496,655.
(60) Provisional application No. 61/167,013, filed on Apr. 6, 2009.
(60) Provisional application No. 61/267,804, filed on Dec. 8, 2009.

(51) Int. Cl. A61B 18/14

(52) U.S. Cl. CPC A61B 18/1492; A61B 2018/00369
USPC 606/48

(57) ABSTRACT

A system and method for resecting a valve in a patient, in which the system includes: a proximal frame and a distal frame opposite the proximal frame, each frame including a plurality of interconnectable frame segments, in which the proximal and distal frames are separated by an adjustable distance and positionable on opposite sides of the valve; an electrode, adjacent to the valve on at least one of the proximal and distal frames, that electrosurgically resects valve tissue; a proximal chamber and a distal chamber coupled to the proximal and distal frames, respectively, that allow passage of fluid and captures at least a portion of resected valve tissue, in which each chamber selectively operates in one of a radially collapsed mode and a radially expanded mode; and a drive system that adjusts the distance between the proximal and distal frames.

### CROSS REFERENCE TO RELATED APPLICATIONS

This application is a continuation-in-part of U.S. patent application Ser. No. 13/928,312 filed on Jun. 26, 2013 and titled “System and Method for Resecting a Valve,” Publication No. US-2014-0012257-A1, which is a continuation of U.S. patent application Ser. No. 12/755,361, filed Apr. 6, 2010, titled “System and Method for Resecting a Valve,” now U.S. Pat. No. 8,496,655, which claims priority to U.S. Provisional Patent Application No. 61/167,013, filed Apr. 6, 2009 and U.S. Provisional Patent Application No. 61/267,804, filed Dec. 8, 2009. Each of these patents and patent applications is herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### TECHNICAL FIELD

This invention relates generally to the surgical field, and more specifically to an improved system and method for resecting a valve in the cardiac surgery field.

### BACKGROUND

Aortic stenosis is an abnormal narrowing of the aortic valve in the heart resulting in decreased cardiac function, which often leads to a deteriorating quality of life for those afflicted by the condition. The most effective conventional treatment for aortic stenosis is aortic valve replacement (AVR) by open heart surgery, which involves removing the existing native aortic valve and implanting an artificial aortic valve, but only a fraction of patients with aortic stenosis are able to undergo this invasive procedure. A less invasive procedure for aortic valve replacement under development is percutaneous or transcatheter AVR (PAVR or TAVI), in which the replacement valve is delivered as part of a stent valve delivery system through a guiding catheter via the vascular system by a transfemoral or directly via a transapical approach.

In percutaneous AVR, a balloon catheter expands to compress the existing native valve to the blood vessel wall prior to insertion of the replacement valve. However, this procedure introduces significant risks, including paravalvular leakage, device embolization, device failure, inadequate valve sizing, induced conduction defects requiring a permanent pacemaker, obstruction of the coronary ostia, distal emboli resulting in stroke and mitral valve injury. Such risks are often increased for certain patients who, for example, have native valves with hardened leaflet calcifications that interfere with the replacement valve stent structure.

Thus, there is a need in the cardiac surgery field to create a system and method for resecting a valve. This invention provides such an improved system and method for resecting a valve.

### SUMMARY OF THE DISCLOSURE

Described herein are apparatus, including device and systems, and methods of using them, for resecting a target cardiac valve.

For example, described herein are apparatus for resecting a target cardiac valve in a patient, comprising: a collapsible distal chamber having a proximally-facing opening; a first annular cutter on the proximally-facing opening comprising a first plurality of nested and interconnectable frame segments; a collapsible proximal chamber having a distally-facing opening; a second annular cutter on the distally-facing opening comprising a second plurality of nested and interconnectable frame segments, wherein the first and second annular cutters are adapted to communicate to cut tissue of the target cardiac valve; an elongate drive shaft extending distally to proximally through the distal chamber and the proximal chamber, wherein the elongate drive shaft is configured to reduce a proximal-to-distal separation between the distal chamber and the proximal chamber; and a tissue engagement region within the distal chamber and the proximal chamber configured to engage and retain the tissue of the target cardiac valve within either or both the distal chamber and the proximal chambers; wherein the distal and proximal chambers are configured to be collapsed around the drive shaft in a delivery configuration and further configured to expand for positioning on either side of the target cardiac valve.

In general, the tissue engagement region may be connected to the drive shaft and may be configured to rotate with the drive shaft, or moved independently of the drive shaft, or may not move. For example, the tissue engagement region may comprise a mesh. In some variations the tissue engagement region comprises a plurality of teeth; the teeth may be coupled to the drive shaft and may rotate with the drive shaft. For example, the tissue engagement region may comprise a plurality of rings coupled to the drive shaft, wherein the rings comprise teeth. The tissue engagement region may comprise a plurality of teeth coupled to the drive shaft intermediate between the distal chamber and the proximal chamber. The first annular cutter comprises a first electrode and the second annular cutter comprises a second electrode, further wherein the first and second electrodes are configured to supply bipolar electrocautery current to electrosurgically resects the target valve tissue.

The distal chamber and/or proximal chamber may includes a plurality of frame arms that collapse radially inwards towards the drive shaft when the chamber is collapsed and open radially outwards when the chamber is expanded. At least one of the proximal and distal chambers may be conical. The proximal and distal chambers may generally allow passage of fluid therethrough.

The apparatus may also include drive system that adjusts the distance between the proximal and distal rings, for example, by rotating the drive shaft (or drive cable). Thus, the drive shaft may be threaded, to allow movement of the proximal and/or distal chambers.

The first plurality of nested and interconnectable frame segments may have angled ends that complementarily mate with the angled ends of adjacent frame segments. In some variations, the first plurality of nested and interconnectable frame segments includes an arc segment having a radially inward angled end and another frame segment having a radially outward angled end that engages with the inward angled end when the distal chamber is expanded.

Any of these apparatus may also include a collapse actuator coupled to the distal chamber and configured to controllably collapse the distal chamber so that it may be withdrawn into a catheter.

An apparatus for resecting a target cardiac valve in a patient may include: a collapsible distal chamber having a proximally-facing opening; a first annular cutter on the proximally-facing opening; a collapsible proximal chamber having a distally-facing opening; a second annular cutter on the distally-facing opening, wherein the first and second annular cutters are adapted to communicate to cut tissue of the target cardiac valve; an elongate drive shaft extending distally to proximally through the distal chamber and the proximal chamber, wherein the elongate drive shaft is configured to reduce a proximal-to-distal separation between the distal chamber and the proximal chamber; and a tissue engagement region comprising a plurality of teeth coupled to the drive shaft between the distal chamber and the proximal chamber and configured to dynamically engage and retain the target cardiac valve tissue within either or both the distal chamber and the proximal chambers; wherein the distal and proximal chambers are configured to be collapsed around the drive shaft in a delivery configuration and further configured to expand for positioning on either side of the target cardiac valve.

Also described herein are methods of treating tissue using any of these apparatus. For example, a method for minimally invasively resecting a cardiac valve may include: positioning a catheter adjacent a target cardiac valve; extending a distal chamber of an apparatus for resecting a target cardiac valve from the distal end of the catheter so that the distal chamber expands on a distal side of the cardiac valve and a proximally-facing opening of the distal chamber faces the cardiac valve; extending a proximal chamber of the apparatus for resecting a target cardiac from the catheter so that the proximal chamber expands on a proximal side of the cardiac valve and a distally-facing opening of the proximal chamber faces the cardiac valve; actuating a drive shaft of the apparatus to shorten the distance between the proximal and distal chambers; engaging the valve tissue with a tissue engagement region on the drive shaft, wherein the tissue engagement region comprises a plurality of teeth; cutting the valve tissue between a first annular cutter on the proximally-facing opening and a second annular cutter on the distally-facing opening to resect the valve tissue; capturing the resected valve tissue in at least one of the proximal and distal chambers; and withdrawing the delivery catheter and resected valve tissue from the vascular system of the patient.

### BRIEF DESCRIPTION OF THE DRAWINGS

FIGS. 1A and 1B are partial views of the system of one embodiment of an apparatus for removing a cardiac valve, showing chambers and cutting frames in their collapsed and expanded modes, respectively.

FIGS. 2A, 2A′, 2B and 2B′ are perspective (2A and 2B) and cross-sectional (2A′ and 2B′) views of exemplary proximal and distal frames of one variation of a cardiac valve removal system.

FIGS. 2C and 2D schematically illustrate frame segments that may be used to form the proximal and distal frames of a device or system for removing a cardiac valve.

FIGS. 3A and 3B are partial schematic illustrations of the frame segments of FIGS. 2A and 2B in a collapsed mode and an expanded mode, respectively, which may form regions (e.g., chambers) of a device or system for removing a cardiac valve.

FIG. 4 is a side view of one variation of a distal chamber of a device or system for removing a cardiac valve.

FIG. 5 is a side view of one variation of the proximal and distal chambers of a device or system for removing a cardiac valve.

FIG. 6 is a partial side view of a frame segment and mesh segment of a device or system for removing a cardiac valve.

FIG. 7 is a cross-section schematic of a frame segment of some variations of a device or system for removing a cardiac valve.

FIG. 8 is a schematic illustration of a drive cable mechanism of some variations of a device or system for removing a cardiac valve.

FIG. 9 is a cross-section schematic of some variations of a drive cable of a device or system for removing a cardiac valve.

FIG. 10 is a partial side view of a power unit and delivery catheter that may be used with some variations of a device or system for removing a cardiac valve.

FIGS. 11-16C are schematics of the steps of the method of a preferred embodiment.

FIG. 11 shows the distal end region of the apparatus in a collapsed configuration and the proximal region.

FIG. 12 illustrates the approach of a catheter (using a guidewire) into a heart in a minimally invasive approach; the catheter may be used to deliver the apparatus of FIG. 11 to replace a diseased or damaged heart valve.

FIG. 13A shows the distal end portion of a delivery catheter delivering an apparatus for removing a cardiac valve; the apparatus may be delivered by the catheter across a target cardiac valve so that the apparatus spans both sides of the valve.

FIG. 13B shows the distal end of the apparatus for removing a cardiac valve inserted across a valve and expanded to form a distal chamber formed by a distal frame on one side of the valve and a proximal chamber formed by a proximal frame on the opposite side of the valve.

FIGS. 13C illustrates actuation of the apparatus by driving the distal and proximal frames toward each other to cut (resect) the valve leaflets positioned between them.

FIG. 14 shows the valve leaflet that is being between the proximal and distal capture chambers.

FIG. 15 illustrates the excised valve within the holding chamber formed between the proximal and distal capture chambers.

FIGS. 16A and 16B illustrate collapsing the distal and proximal capture chambers to capture the excised tissue within one or both of them, so that the apparatus can be withdrawn proximally using a catheter (e.g., the catheter shown in FIG. 16A).

FIG. 16C illustrates withdrawal of the apparatus using the catheter, removing the excised valve as well.

FIG. 17A schematically illustrates another variation of an apparatus for removing a cardiac valve that may be used as described herein. FIG. 17A is a side perspective view that illustrates gripping or tissue-engaging members (configured as toothed rings in this example) within the apparatus.

FIG. 17B shows another perspective view from a different angle, of the apparatus of FIG. 17A.

FIG. 17C is a partial bottom perspective view of the distal end of the apparatus of FIG. 17A.

FIG. 17D illustrates a section though the region of the apparatus between proximal and distal capture chambers, showing an internal excised-tissue engaging region and the distal capture chamber in an expanded configuration.

### DESCRIPTION OF THE PREFERRED EMBODIMENTS

The following description of preferred embodiments of the invention is not intended to limit the invention to these preferred embodiments, but rather to enable any person skilled in the art to make and use this invention.

In general, these apparatuses (e.g., devices and systems) may include a proximal capture region (chamber) and a distal capture region at the distal end region of the apparatus. The proximal and distal chambers may each be formed of a separate frame, and both are configured to be collapsed from an expanded chamber. In the expanded configuration the perimeters of the facing surfaces of the distal chamber and the proximal chamber may be adapted to cut tissue (e.g., resect a valve) positioned between them. Further, the apparatus may generally be adapted to shorten the distance between the facing surfaces of the distal and proximal chambers may be moved closer together to trap or capture the tissue being cut. Thus, the distal chamber may be driven proximally and/or the proximal chamber may be driven distally to either cut and/or capture tissue between the two, and any of the apparatuses described herein may include a drive shaft (or shafts) for driving the proximal and/or distal chambers together to cut and/or capture tissue within the chambers of the apparatus.

In the collapsed configuration the apparatus may be fitted into a catheter (e.g., delivery catheter) from which it may exit to expand. Thus, a system may include a delivery catheter and/or guidewire. The apparatus may also include a lumen or passage through (e.g., a central or mid-line lumen) for passing and operating a guidewire.

The apparatus may be biased in an expanded configuration (e.g., with the distal and proximal chambers expanded) so that releasing the apparatus from within a catheter may automatically expand the distal and proximal chambers on either side of a valve. In some variations the distal chamber may be separately expanded (and/or collapsed) relative to the proximal chamber, which may assist in positioning the apparatus on either side of a valve to be resected. The distal and/or proximal chambers may be adapted to controllable collapse, e.g., using an actuator, such as a collapse wire, ring, string, or the like.

Any of the apparatuses may be adapted to engage, grasp or grip tissue to be cut and/or that has already been cut. For example, in some variations the apparatus includes one or more tissue engagement regions (e.g., teeth) on the drive shaft (or shafts) between the chambers. The tissue engagement regions may be dynamic, meaning that they can be moved (e.g., rotated, laterally displaced, opened/closed, etc.). For example, movement of the drive shaft to close the gap between the distal and proximal chambers formed in the expanded configuration may move the tissue engagement region(s) within the apparatus positioned between the chambers so that the tissue engagement region(s) engage (e.g., snag, grasp, grab, hold, etc.) tissue within the chambers. This may prevent captured tissue from being released even when withdrawing the apparatus proximally.

### 1. Apparatus (Devices and Systems) for Resecting a Valve

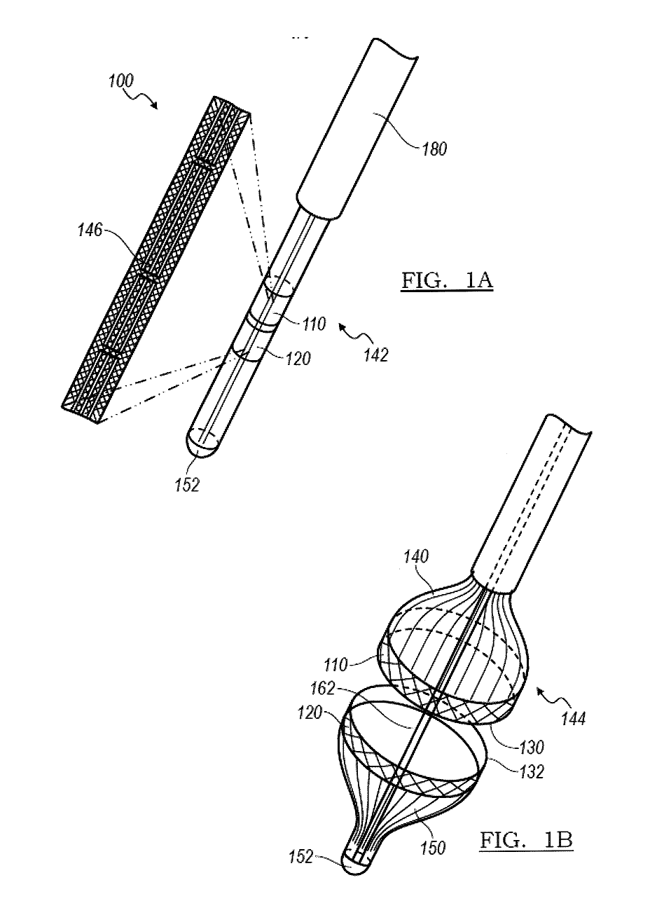

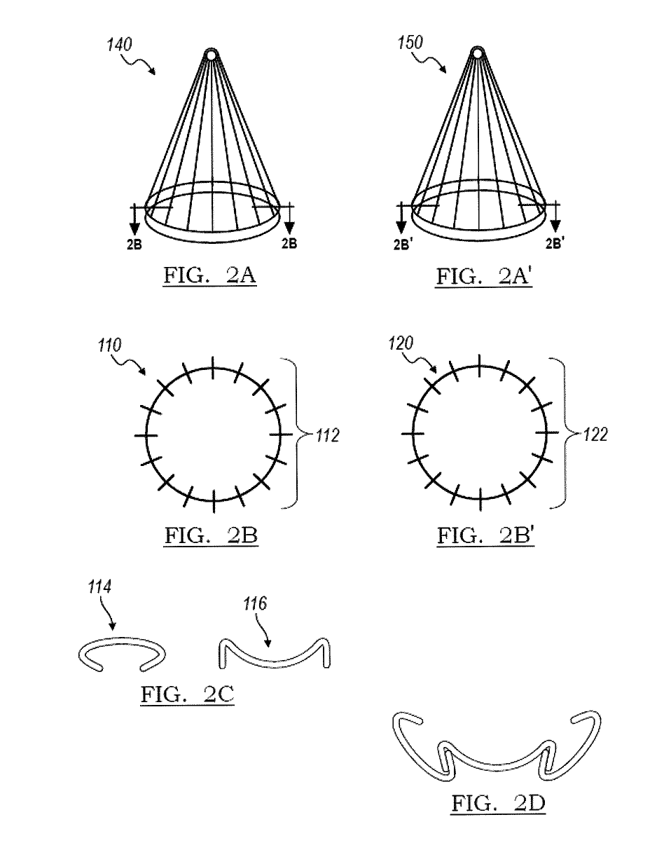

As shown in FIGS. 1 and 2, a system 100 for resecting a valve in a patient may include: a proximal frame 110 formed by a first plurality of interconnectable arc segments 112 and a distal frame 120 formed by a second plurality of interconnectable arc segments 122 opposite the proximal frame no, in which the proximal and distal frames are separated by an adjustable distance 124 and positionable on opposite sides of the valve; at least one electrode on at least one of the proximal and distal frames that electro surgically resects valve tissue; a proximal chamber 140 coupled to the proximal frame and a distal chamber 150 coupled to the distal frame 120, in which the proximal and distal chambers allow passage of blood or other fluid and capture at least a portion of the resected valve tissue; and a control system 160 including a drive shaft (cable 162) that adjusts the distance 124 between the proximal and distal frames. Each of the proximal and distal chambers preferably operates in at least one of the following modes: a radially collapsed mode 142 in which the plurality of arc segments are nested, and a radially expanded mode 144, in which the plurality of arc segments are expanded relative to each other and interconnect to form a generally circular shape. The system 100 preferably further includes a delivery catheter 180 that delivers the proximal and distal frames and chambers to and from the valve, and the control system 160 preferably controls operation of the distal and proximal frames and distal and proximal chambers. The system 100 is preferably used to excise or resect the aortic valve of a human or other animal patient prior to aortic valve replacement, in particular percutaneous aortic valve replacement (PAVR). Preferably, the system removes all of the valve tissue and captures all of the removed valve tissue. However, in some embodiments, the system may remove only a portion of the valve tissue and/or capture only a portion of the valve tissue. For example, the system may be accompanied by another instrument that is operated to remove another portion of the valve tissue and/or capture another portion of the valve tissue, such that the system and the instrument (or multiple instruments) cooperate to completely excise and capture all of the valve tissue. In an alternative embodiment, the system is integrated with a system for aortic valve replacement in a single instrument, such as an instrument that incorporates a replacement valve with the system for minimally invasive electrosurgical valve excision. The system 100 may be used in a transfemoral approach, a transapical approach, or any other suitable approach to the aortic valve. The minimally invasive approach of the system to excise the valve complements the minimally invasive nature of percutaneous aortic valve replacement, and does not require the increased risk of cardiopulmonary bypass. However, the system may alternatively be used to excise any cardiac valve or other valve in the cardiovascular system, or any suitable tissue accessible by catheter. Entire excision of native valve tissue prior to valve replacement creates a known and consistent smooth annulus in which the replacement valve will be placed, which decreases or eliminates risks such as paravalvular leakage that are associated with a replacement valve needing to adapt to the native valve.

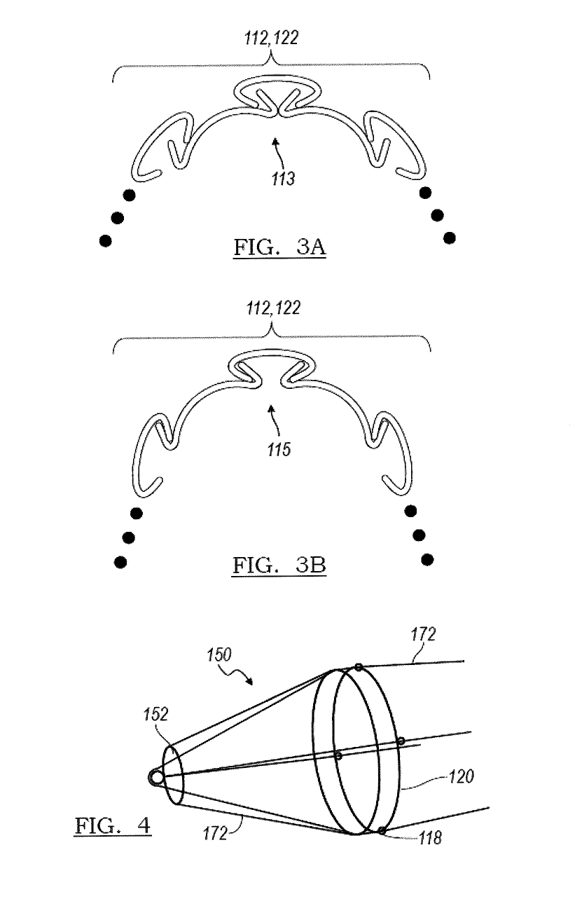

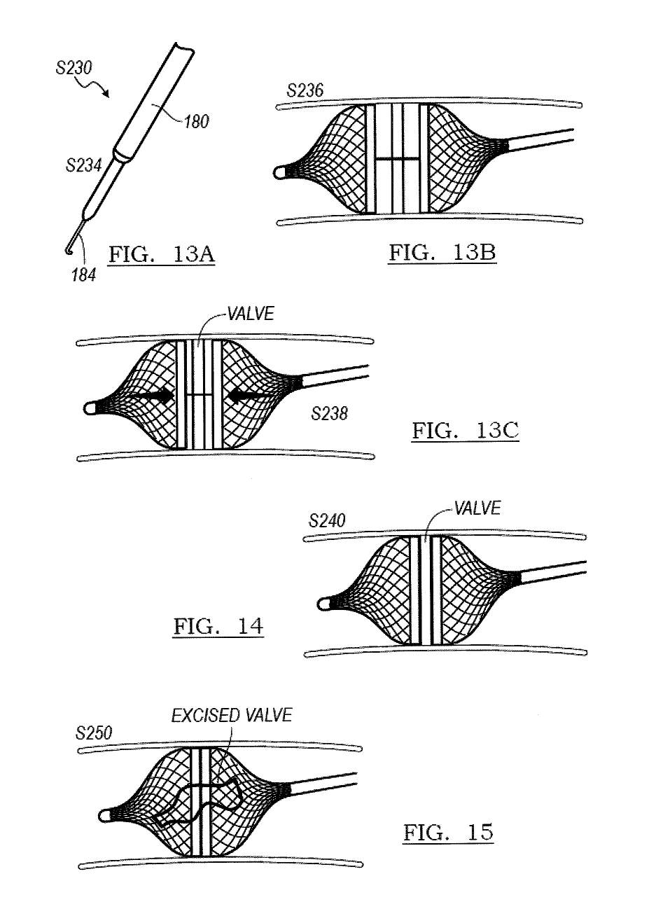

The proximal and distal frames 110 and 120, respectively, of the system function to provide structural support for the electrodes and the proximal and distal chambers. In some embodiments, the system may include only one frame, or may have three or more frames for any suitable number of electrodes and/or chambers. As shown in FIG. 14, the proximal and distal frames are preferably adjacent to one another, and face opposite to one another such that prior to excision of the valve, the valve tissue to be excised is positioned between the proximal and distal frames. When properly positioned in a transfemoral approach, the distal frame 120 is preferably located inferior to the aortic valve and the proximal frame 110 is preferably located superior to the aortic valve. Alternatively, when properly positioned in a transapical approach, the distal frame 120 is preferably located superior to the aortic valve and the proximal frame 110 is preferably located inferior to the aortic valve. As shown in FIGS. 3A and 3B, each frame preferably operates in at least two modes: a collapsed mode 113 and an expanded mode 115, which correspond to the collapsed and expanded modes 142 and 144, respectively, of the proximal and distal chambers. In the collapsed mode 113, the proximal or distal frame preferably radially collapses into a compact shape that can fit into the delivery catheter for positioning in and withdrawal from the patient, as well as pass through the opening of the native valve. In the expanded mode 115, the proximal or distal frame preferably radially expands to a full, substantially circular shape for valve tissue excision. The proximal and distal frames each preferably have a diameter of approximately 6 mm in the collapsed mode and approximately up to 26 mm in the expanded mode, but may alternatively be any suitable size and/or shape in either mode. For example, smaller dimensions may be appropriate for a pediatric patient. The proximal and distal frames preferably have a diameter ratio of approximately 1:4 in the collapsed mode relative to the expanded mode, but may alternatively have any suitable ratio. The proximal and distal frames are preferably identical in size, but may alternatively be different sizes and/or shapes.

As shown in FIGS. 2A-3B, each of the proximal and distal frames 110 and 120 forming the chambers may include a plurality of frame segments 112 and 122, respectively. The frame segments are preferably interconnecting arcuate pieces that form the frame structure. The frame segments are preferably approximate arc segments of a generally round ring. As shown in FIG. 3A and 3B, each pair of adjacent arc segments preferably has complementary angled ends that unlock to enable the collapsed mode and lock to enable the expanded mode of the frame. More preferably, as shown in FIG. 2C, within a pair of adjacent frame segments in a plurality of frame segments, one arc segment preferably has ends 114 that are angled radially inwards, and the other adjacent arc segment preferably has ends 116 angled radially outwards, such that an inward angle end of one piece and an outward angle end of an adjacent piece engage to enable the expanded mode of the frame, and disengage to enable the collapsed mode of the frame. However, the frame segments may alternatively be any suitable length and shape, depending on the desired size and shape of the frame and/or interconnect in any suitable manner, such as with pins and slots. As an example, the frame segments may be shaped such that the frame in its expanded mode is elliptical or square. The number of frame segments that form the frame is a predetermined number, preferably based on the desired frame size that is dependent on the geometry of the intended replacement valve. In its expanded mode, the proximal frame 110 and/or distal frame preferably has a size and shape similar to that of the replacement valve.

Although this example shows frame segments, the frames forming the proximal and distal capture regions may be formed of wire, mesh, or the like, which may be fixedly joined relative to each other (e.g., at intersecting regions). Thus, a proximal or distal frame (and therefore a proximal or distal chamber) may be made of a wire material that is configured to for the expanded chamber shapes (e.g., bell-shapes, cone-shapes, funnel-shapes, tube-shapes, etc.) for both the proximal and distal chambers. As used herein, a frame portion of a chamber may include both the perimeter cutting region that faces the other chamber or the struts/links forming the rest of the chamber shape.

As shown in FIG. 4, a proximal and/or distal frame may include at least one eyelet 118 or a plurality of eyelets 118 that are distributed around at least one of the frames. The eyelets 118 may function to provide guidance for control wires that control a collapse or compression ring 152 on the distal chamber 150. As further described below, the collapse/compression ring 152 may help the distal frame 120 and therefore the distal chamber 150 transform from the expanded mode into the collapsed mode, and/or transform from the collapsed mode to the expanded mode. In a preferred embodiment, at least one of the proximal and distal frames preferably includes four eyelets equally distributed around the circumference of the frame at 90 degrees from each other when the frame is in its expanded mode, but may alternatively include any suitable number of eyelets arranged in any suitable arrangement. Alternatively, the proximal and/or distal frame may include channels, hooks, or any suitable guidance for the compression ring control wires.

In some variations, the cutting of tissue (e.g., valve tissue) between the proximal and distal chambers is done electrically. For example, the frame region may include electrodes. The electrodes may function to cut through valve tissue. The system preferably includes at least one proximal electrode 130 on the proximal frame 110 and at least one distal electrode 132 on the distal frame, preferably on the frame segments around the perimeter of each frame, such that during valve excision, the valve tissue that is positioned between the opposing distal and proximal frames is approached by the electrodes from two sides, thereby reducing required cutting time. Alternatively, only one of the proximal and distal frames may include an electrode. The electrodes 130 and 132 preferably include electrode segments coupled to frame segments that collapse and expand correspondingly to the proximal and distal frames, although the specific geometry and arrangement of the electrodes may depend on the geometry of the frame segments and/or the anatomy of the valve tissue to be cut. The electrodes may, however, be of unitary construction such as being ring-shaped. The electrodes 130 and 132 preferably perform bipolar electrosurgery to cauterize and cut through the valve tissue, but may alternatively be any suitable kind of electrode capable of cutting through tissue. The electrodes are preferably powered with electrocautery current supplied by a bipolar electrosurgical generator, but the electrodes may additionally and/or alternatively be powered by any suitable radiofrequency or other suitable energy source. The electrodes are preferably similar to conventional bipolar electrosurgical electrodes, and are preferably made of a conductive material such as gold or silver to decrease the amount of time required to cut tissue. In an alternative embodiment, one of both of the frames may additionally and/or alternatively include other means for cutting through tissue, such as blades, lasers, or RF energy.

The proximal and distal chambers 140 and 150 of the system function to contain the valve tissue after valve excision. The proximal chamber 140 is preferably coupled to the proximal frame 110, and the distal chamber 150 is preferably coupled to the distal frame 120, thus the proximal frame forms a part of the proximal chamber (e.g., the perimeter region facing the distal chamber). The system may alternatively include more or fewer chambers. As shown in FIG. 13B, the proximal and distal chambers 140 and 150 are preferably opposed to one another and can be positioned on opposite sides of the native valve before valve excision, with open ends that face each other across the native valve. After the valve tissue is cut, the proximal chamber and/or distal chamber captures the excised or resected valve tissue. Similar to the proximal and distal frames, each chamber preferably operates in at least one of two modes: a collapsed mode 142 to fit within the delivery catheter and through the native valve before valve excision, and an expanded mode 144 to facilitate capture of the excised or resected valve tissue. The collapsed and expanded modes 142 and 144 of the chambers preferably correspond to the collapsed and expanded modes 113 and 115 of the frames, respectively, in that the frames and chambers are preferably in their collapsed modes simultaneously and in their expanded modes simultaneously. In the collapsed mode, each chamber preferably includes an interior space large enough to accommodate valve tissue, for containing the valve tissue during withdrawal from the patient through the delivery catheter. The proximal and distal chambers may be identical in size, such that their facing open ends are equal in size in the expanded mode, or one chamber may be slightly smaller than the other such that the open end of the smaller chamber nestles slightly within the open end of the larger chamber. In some variations, each of the proximal and distal chambers includes a plurality of frame arms and/or a mesh filter. In some variations the frame arms may themselves form a mesh or mesh-like filter. The mesh may operate as a tissue engagement/tissue engaging region or portion of the apparatus.

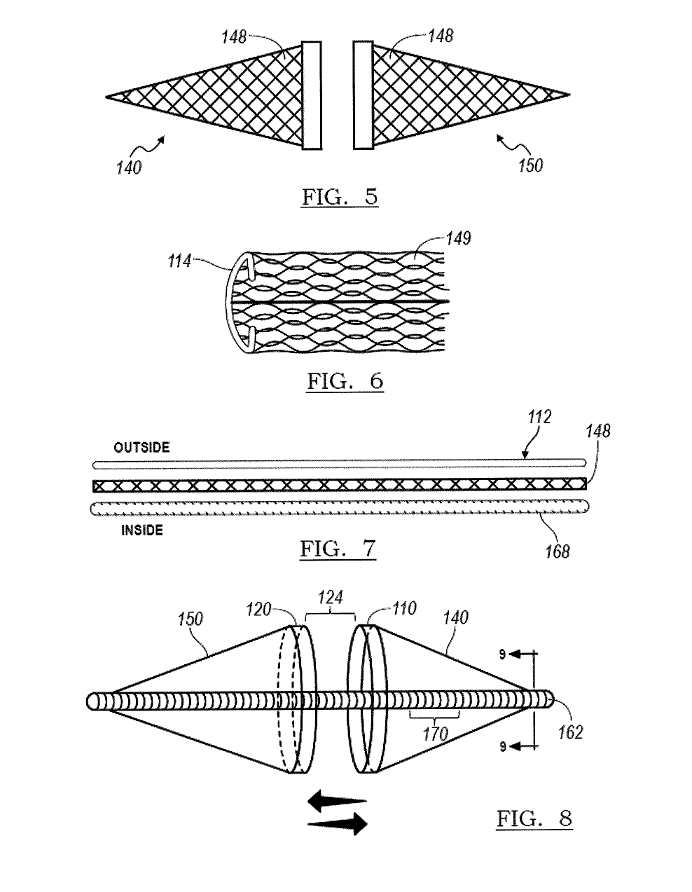

The plurality of frame arms 146 may provide structural support for the chamber, and deploy the chamber and frame into the expanded mode. In the collapsed mode of the chamber, the frame arms may collapse radially inwards, and in the expanded mode of the chamber, the frame arms preferably expand radially outwards. As shown in FIGS. 4 and 5, in the expanded mode of the chamber, the frame arms may define a generally conical shape, but may alternatively define a frustoconical shape, a bell shape (shown in FIG. 1), a cylinder, or any suitable shape may be formed. The plurality of frame arms 146 may self-expand, similar to conventional Nitinol stents. However, the frame arms 146 may additionally and/or alternatively expand through actuated means such as balloon inflation, or any suitable mechanism. Thus, the frame arms 146 forming a chamber may be made of Nitinol, similar to conventional stents, but may be made of another shape memory alloy or any suitable biocompatible material (including stainless steel, and the like). The frame arms may be made in a manufacturing process similar to that of stents, such as laser cutting. The frame arms of the proximal chamber are preferably identical to those of the distal chamber, but the frame arms of the distal and proximal chambers may alternatively be different. In alternative embodiments, the proximal and/or distal chamber may lack a plurality of frame arms, such that the mesh filter is structural supported by only the proximal and/or distal frame around its perimeter, and/or another suitable attachment point.

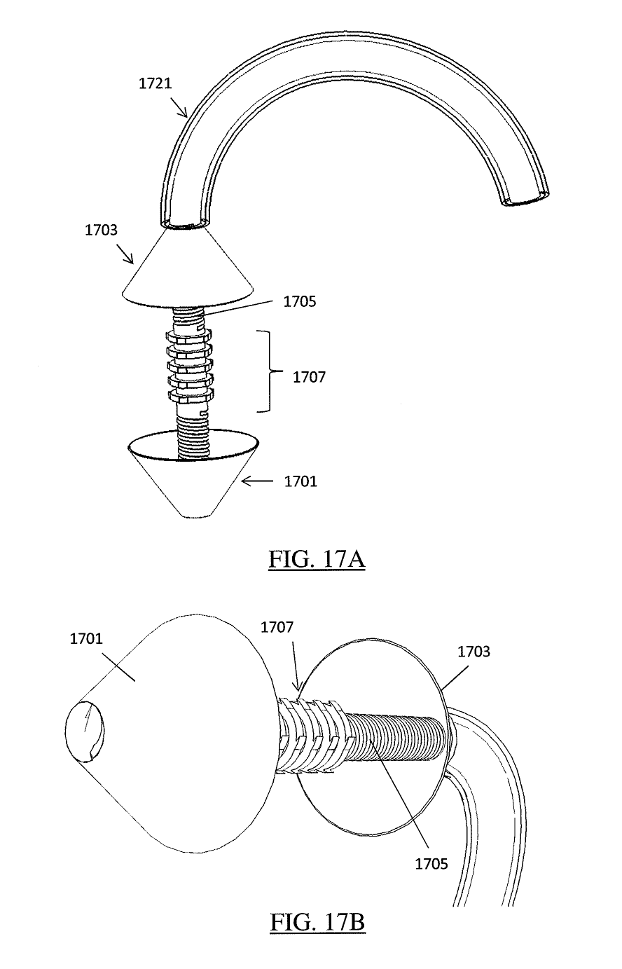

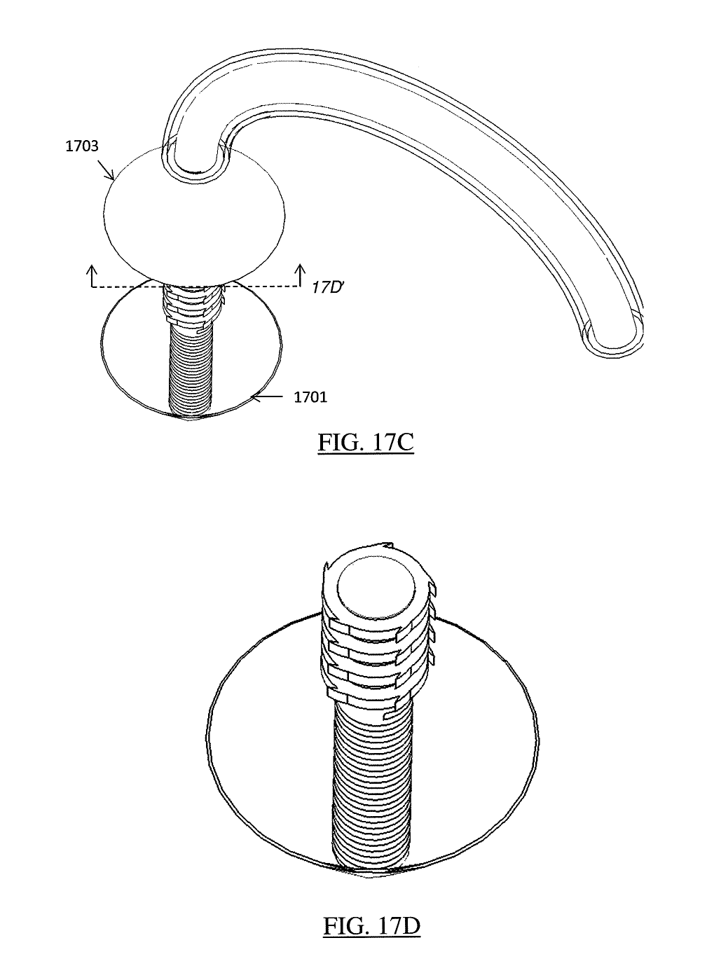

In some variations, the mesh filter 148 (or the frame arms forming the ‘mesh’) preferably allows passage of fluid (e.g., blood, flushing fluid, etc.) and may capture the excised valve tissue during and after valve excision. The mesh filter 148 may also capture other particles, such as calcium or other mineral deposits on the tissue and/or separated from the tissue. In some embodiments, the mesh filter 148 may include a single net-like piece that is coupled to some or all of the frame segments. In other embodiments, the mesh filter may include multiple overlapping mesh segments 149, such that each mesh segment is coupled to a frame segment (shown in FIG. 6) and overlaps with adjacent mesh segments a sufficient amount as to avoid gaps between mesh segments. As shown in FIG. 7, the mesh filter 148 in some variations is attached to the inside surfaces of the frame arms 146 of the chamber, thereby substantially forming the walls of the chamber. Alternatively, the mesh filter 148 may be attached to the exterior surfaces of the frame arms 146 of the chamber, or any other suitable surface. The mesh filter 148 is preferably a mesh membrane with a mesh size of approximately 55 microns, but may have any suitable size mesh to allow blood to pass through the mesh filter, while preventing passage of and capturing excised valve tissue particles. The mesh membrane is preferably made of polyethylene, but may alternatively be made of any flexible mesh capable of being easily compacted in the collapsed state of the chamber. In alternative embodiments, the chamber may additionally and/or alternatively include other means to engage, contain and/or excise the valve tissue from the area following valve excision, such as suction or other tissue engagement regions (e.g., toothed regions). In any of these variations, the tissue engagement region may be coupled to inner structures within and/or between the chambers, including the drive shaft (drive cable). For example, a mesh material may be attached to the drive cable so that movement of the drive cable may move the mesh material within the region between the chambers to help engage the tissue that is cut or being cut. Another example of a dynamically operable tissue engagement region is described below in reference to FIGS. 17A-17D.

The distal chamber 150 may be configured to controllable collapse. For example, the distal chamber may include an actuator for collapsing the distal chamber (including the distal frame), such as compression ring 152 that helps the distal chamber and distal frame transform from their expanded modes into their collapsed modes. The compression ring 152 may slidingly engage with the frame arms 146 of the distal chamber 150 to transition the distal chamber 150 from the expanded mode 142 to the collapsed mode 144. In this example, the compression ring 152 includes a single central hole or lumen that slides over the exterior of the distal chamber 150, but the compression ring may alternatively include multiple holes or lumens. For example, in an embodiment in which the distal chamber 150 includes longitudinal frame arms that expand like an umbrella, the compression ring may have a compactly arranged plurality of holes (such as in a circle), in which each hole is slidingly engaged with a frame arm such that when the compression ring is drawn over the distal chamber, the frame arms are constrained to collapse relative to one another. As shown in FIG. 4, the compression ring is preferably drawn from the distal end of the distal chamber towards the proximal end of the distal chamber by at least one compression ring control wire 172. When the compression ring control wires 172 are pulled, the compression ring slides over the distal chamber in a proximal direction to help transform the distal chamber and distal frame from their expanded modes into their collapsed modes, particularly after valve excision. The compression ring may additionally and/or alternatively slide over the distal chamber in a distal direction to help transform the distal chamber and distal frame from their collapsed mode to their expanded modes. The compression ring control wires 172 are approximately equally distributed around the perimeter of the distal frame and distal chamber, guided through eyelets 118 on the distal frame and/or proximal frame 110 towards the compression ring. The system preferably includes four compression ring control wires, but may alternatively include any suitable number of compression ring control wires 172. Alternatively, the distal chamber may transition to the collapsed mode with any suitable mechanism or manner. When the distal chamber transitions to the collapsed mode, the proximal chamber preferably correspondingly returns to its collapsed mode as the proximal chamber is withdrawn, proximal pointed end first, back into the delivery catheter 180 after excision of the valve tissue, but the proximal chamber may additionally and/or alternatively include a compression ring or another similar structure to help return to its collapsed mode.

In a preferred embodiment, the system further includes a delivery catheter 180 that delivers the proximal and distal frames and chambers to the valve. The delivery catheter 180 may be similar to PAVR delivery catheters and other catheters that access the aortic valve, such as that described in U.S. Pat. No. 4,777,951 entitled, “Procedure and catheter instrument for treating patients with aortic stenosis”, which is incorporated in its entirety by this reference. The delivery catheter 180 preferably accesses the vascular system by entering through a femoral artery, but may alternatively access the vascular system through any suitable artery, vein or other point of entry. Once within the aortic arch, the delivery catheter is preferably advanced until the collapsed distal chamber and distal frame pass through the aortic valve, and the aortic valve is properly positioned between the distal frame and the proximal frame 110 for valve excision. The delivery catheter may enable a second catheter to simultaneously access the aortic valve. This simultaneous access to the aortic valve allows, for example, a PAVR catheter to enter the vascular system through the second, unused femoral artery of the patient so that valve replacement may be performed very soon after native valve excision is completed, shortening the total surgery time and possibly reducing complications.

As shown in FIG. 10, the proximal portion of the delivery catheter 180 may include a hemostatic valve 182 such as a Tuohy-Borst hemostatic valve, which are commonly used in percutaneous procedures to provide a fluid seal that prevents “back-bleeding”, or blood leakage or loss through the catheter. The proximal end of the delivery catheter may additionally and/or alternatively include any suitable mechanism to prevent blood leakage, such as a gasket or plug.

The control system 160 controls the operation of the distal and proximal frames and chambers, such as adjusting the distance 124 between the proximal and distal frames. The control system 160 preferably includes a drive system with a drive cable 162 that moves the distal and proximal frames relative to one another, a central guidewire 184 that provides control of the delivery catheter, the set of compression ring control wires 172 that move the compression ring 152, a set of current supply cables 174 that provide current to the electrodes, and a power unit 190 that supplies power to the system and provides an operation interface for the control system. The control unit is preferably handheld by a user outside the body of the patient, and may have finger grooves 197 and/or other contours to promote an ergonomic feel.

The drive cable 162 of the drive system functions to move the distal and proximal frames relative to one another. The drive cable 162 preferably selectively opens and closes the gap distance 124 between distal and proximal frames in which the native valve lies prior to valve excision. The drive cable preferably includes external threads 170 and runs coaxially inside the delivery catheter 180 and, as shown in FIG. 8, is preferably coaxial with the proximal chamber 140, the proximal frame 110, the distal frame 120, and the distal chamber 150. In one variation, the distal chamber 150 is fixed to the drive cable 162 and the proximal chamber 140 is held stationary and not fixed to the drive cable 162, such that rotation of the drive cable 162 rotates the distal chamber 150 and moves the distal chamber towards and away from the proximal chamber, thereby opening or closing the gap 124 between the distal and proximal frames (depending on the direction of rotation). Alternatively, the distal chamber may be held stationary and the proximal chamber may be fixed to the drive cable. In a second variation, the exterior of the drive cable and either the proximal chamber or distal chamber include reverse (left-hand) threads, such that rotation of the drive cable causes the distal and proximal frames to move toward and away from one another, similar to a turnbuckle. However, the drive system may additionally and/or alternatively include magnets and/or any suitable mechanism located in any suitable location to move the distal and proximal frames relative to one another.

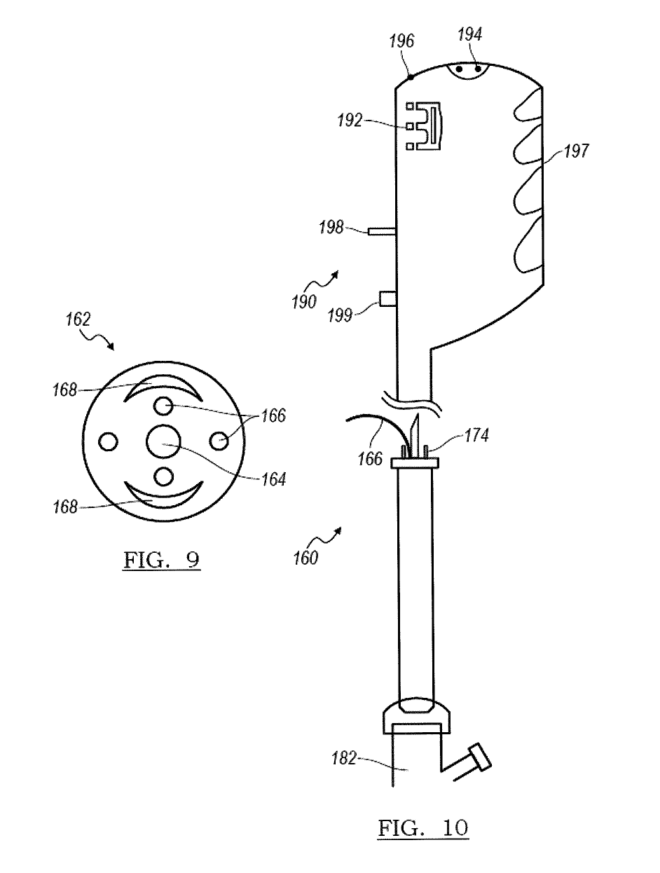

The drive cable 162 of the drive system preferably includes a plurality of lumens that contain and guide the central guidewire, the compression ring control wires, and the current supply cables within the drive cable. As shown in FIG. 9, the drive cable preferably includes a central lumen 164 located at the center of the drive cable to accommodate the central guidewire 184, four approximately equally distributed lumens 166 for the compression ring control wires 172, and a two crescent-shaped lumens for the current supply cables 168. The drive cable 162 may additionally and/or alternatively include any suitable number, shape, and size of lumens that accommodate any suitable wires or objects that pass from the power unit to the distal end of the distal chamber. The central portion of the drive cable that includes the lumens may be independent of the exterior thread portion, such that as the drive cable rotates, the central portion of the drive cable does not rotate, thereby preventing the wires and cables passing through the lumens from twisting with the rotation. As an example, the central portion of the drive cable may be a rod that spins freely within an externally-threaded tube, aided by lubricants or a suitable mechanism.

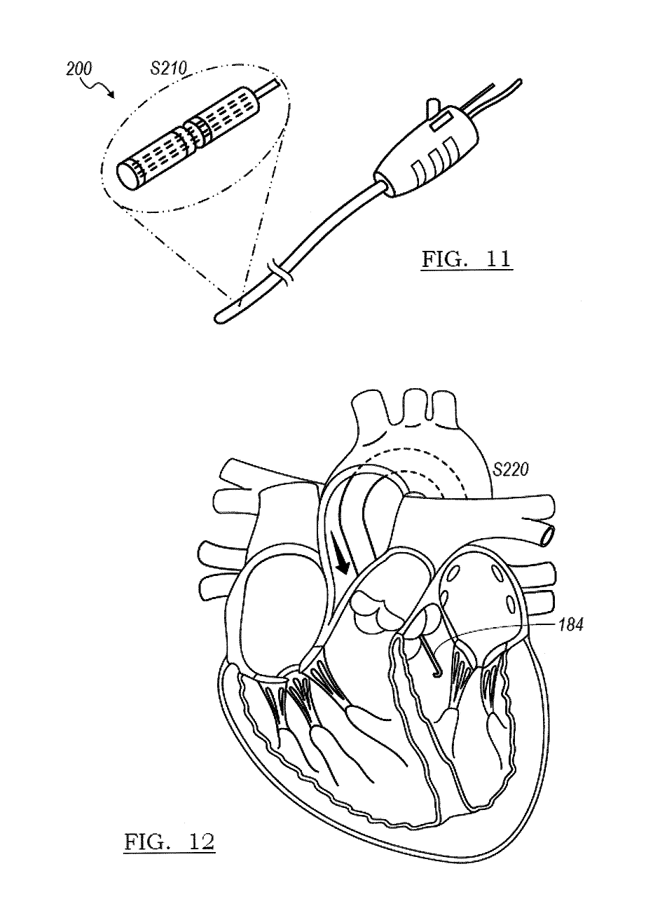

As shown in FIGS. 12 and 13, the central guidewire 184 of the control system functions to provide control of the delivery catheter and guide the delivery catheter through the vascular system to the valve. The central guidewire 184 is preferably similar to those known to one skilled in the art, such as the guidewire described in U.S. Pat. No. 4,777,951 referenced above, but may be any suitable kind of guidewire. As shown in FIG. 9, the central guidewire preferably pass axially within a central lumen of the drive cable, from the power unit to the distal end of the distal chamber.

As described above, the compression ring control wires 172 of the control system function to move the compression ring that transitions the distal frame 120 and chamber from the expanded mode to the collapsed mode. As shown in FIG. 9, the compression ring control wires preferably pass through a plurality of lumens 166 in the drive cable. As shown in FIG. 5, the compression ring control wires 172 preferably pass axially within designated lumens of the drive cable, from the power unit 190 to the compression ring. Near the power unit end, the compression ring control wires 172 may be braided into a single control cable and coupled to a control cable lever 198 traveling within a groove on the power unit 190. Pulling the lever proximally preferably retracts the set of compression ring control wires, thereby sliding the compression ring over the distal chamber to collapse the distal chamber. However, the compression ring control wires may alternatively be selectively pulled in any suitable manner, such as with a mechanism involving springs, notches, or catches. The compression ring control wires are preferably made of stainless steel, but may alternatively be made of any high tensile, biocompatible material or suitable material. The control wires may be operated on a pulley system, another continuous loop mechanism, or other suitable bidirectional control mechanism, such that pulling the compression ring control wires 172 in one direction compresses or retracts the distal chamber and pulling the compression ring control wires 172 in an opposite direction re-expands the distal chamber, thereby enabling transformation between the collapsed and expanded modes. A similar setup with another compression ring and control wires may additionally and/or alternatively be coupled to the proximal chamber to transform the proximal chamber from the expanded mode to the collapsed mode and/or from the collapsed mode to the expanded mode.

The current supply cables 174 of the control system function to supply current to the electrodes 130 and 132 that are used to excise the native valve tissue. The current supply cables 174 preferably supply bipolar electrocautery current or other suitable energy source to the proximal and/or distal frames. As shown in FIG. 9, the current supply cables preferably pass axially within designated lumens of the drive cable, from the power unit to the inner edges of the distal and proximal frames as shown in FIG. 7. The control subsystem preferably includes at least two current supply cables, one serving as an active path and one serving as a return path for the current. The control subsystem may, however, include any suitable number of current supply cables, such as a pair of current supply cables for each separate frame. The current supply cables 174 are preferably insulated with plastic or any suitable insulation, to help prevent unintentional electrical conduction between the current supply cables and portions of the drive cable, including other wires and cables passing through lumens within the drive cable 162.

The power unit 190 of the control system functions to supply to the system and provides an operation interface for the control system. As shown in FIG. 11, the power unit preferably includes a drive unit 192 to control the drive cable, a coupling port 194 to connect to an electrocautery power supply, and a guidewire lumen port 196 that facilitates an “over the wire” delivery catheter insertion approach, a control cable lever 198, and a power switch 199 that turns the system on and off.

The drive unit 192 for controlling the drive cable 162 preferably includes controls such as a knob, switch, or lever that manipulates the drive cable in forward and reverse directions, and in a neutral operation. The drive unit 192 may include a motor, gears, pulleys, and/or any suitable drive control system. The drive unit is preferably battery-operated, but may additionally and/or alternatively be connected to an external power source. The drive unit 192 preferably operates with selected specified revolutions per minute (RPM), resulting in selected speeds at which the distal frame 120 and proximal frame move towards or away from one another. The drive unit may alternatively be external to the power unit, and connected to the drive cable with any suitable drivetrain system.

The coupling port 194 preferably includes contacts that connect to an external generator that generates electrocautery current, preferably bipolar electrocautery current. The specific frequency of current that the coupling port is preferably in the radiofrequency band, but may alternatively be any suitable frequency depending on the specific application.

The guidewire lumen port 196 preferably extends through the entire length of the power unit. As is known to one skilled in the art, in an “over the wire” delivery catheter insertion approach, the guidewire 184 is preferably inserted through the guidewire lumen and into the vascular system. The delivery catheter 180 is then inserted into the vascular system like a sleeve over the guidewire 184, following the trajectory of the guidewire in the vascular system.

The control cable lever 198, as mentioned above, provides selective manipulation of the compression ring control cables 172. The control cable lever 198 preferably moves within a groove on the power unit, and preferably provides tactile feedback such as a catch or latch that signals full retraction of the compression ring control cables, which indicates successful collapse of the distal chamber and distal frame 120. The feedback may alternatively and/or additionally include visual feedback (such as a light) and/or audio feedback (such as a click) that indicates the distal chamber and distal frame 120 are in their collapsed modes. The control cable lever 198 may include a similar feedback to indicate that the distal chamber and distal frame 120 are in their expanded modes. The control cable lever 198 may alternatively and/or additionally include any suitable mechanism, such as a slide switch, knob and or safety lock system.

### 2. Method of Resecting a Valve in a Patient

In general, the methods described herein may include positioning an apparatus having a collapsible distal chamber that is positioned opposite a collapsible proximal chamber so that an annular distal cutting region (e.g., frame) on the distal chamber is opposite a complimentary annular proximal cutting region (e.g. frame) on the proximal chamber. This step may be accomplished by passing a catheter to or adjacent the valve to be cut using a minimally invasive technique and passing the apparatus in a collapsed configuration (with the distal and proximal chambers and/or frames collapsed) so that it spans the valve with the distal chamber/frame on one side of the valve and the proximal chamber/frame on the other side of the valve. The distal chamber/frame may be expanded either during or after it is positioned on the opposite side of the valve from the proximal chamber/frame. For example, the distal chamber may be positioned on one side of the valve by pushing it from a catheter in which it is constrained in a collapsed configuration, allowing it to expand as it leaves the catheter. The catheter can be further withdrawn to expand the proximal chamber/frame on the opposite side of the valve. Thereafter, the proximal and distal chambers/frames may be moved nearer each other so that the valve is positioned between them. This step may also include engaging the tissue to be removed with a tissue engagement region within and/or between the two chambers. For example, the drive cable/shaft may be actuated to rotate and drive the distal and proximal chambers closer together; in variations in which the tissue engagement region is coupled to the drive shaft and can be moved by the drive shaft, the tissue engagement region may also be actuated to engage the tissue. The tissue may be cut by the region forming the perimeter or mouth(s) of the distal and proximal frames of the chambers either before, during or after actuating a drive member to bring the distal and proximal chambers closer together. Cut tissue may be engaged by the tissue engagement region that can help retain the cut tissue (including debris) within the region between the distal and proximal chambers. Once the tissue is cut, the apparatus can collapsed and/while withdrawing it proximally to remove it from the body and withdrawing the cut material.

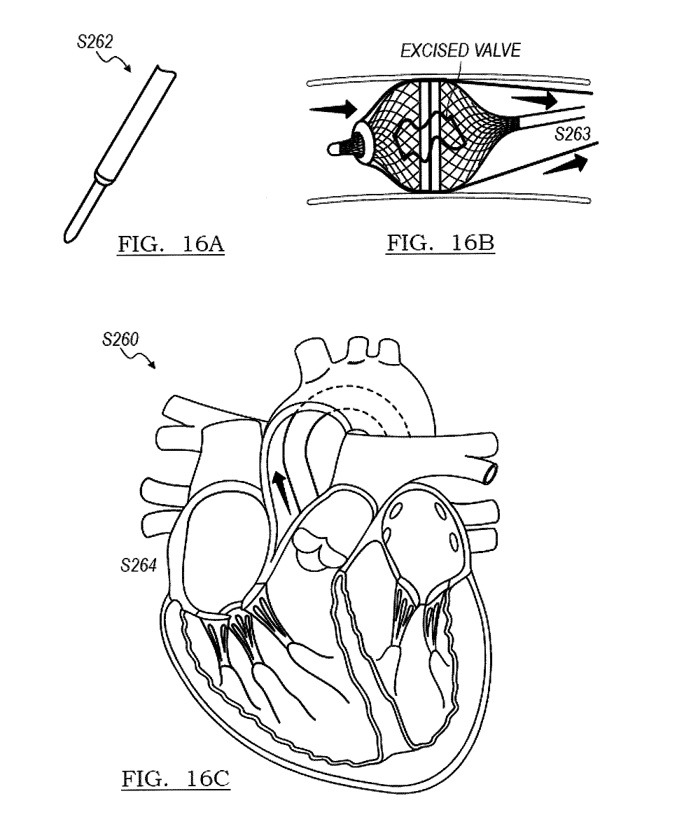

FIGS. 11-16C illustrate one method of resecting a heart valve. In FIGS. 11-16C, the method 200 of resecting a valve in a patient preferably includes the steps of: S210, which may include providing an apparatus including a proximal frame and a distal frame opposing the proximal frame, wherein each of the proximal and distal frames includes a plurality of interconnectable frame segments, an electrode on the proximal and/or distal frame that is adapted to electrosurgially resect valve tissue, and a proximal chamber and distal chamber coupled to the proximal and distal frames, respectively, that are selectively operable in a radially collapsed mode and a radially expanded mode; S220, which includes navigating a delivery catheter through the vascular system of the patient, in which the proximal and distal chambers are in the collapsed mode and sheathed within the delivery catheter; S230, which includes positioning the proximal and distal frames on opposite sides of the valve; S40, which includes activating the electrode to resect the valve tissue; S50, which includes capturing the resected valve tissue in the proximal and/or distal chambers; and S260, which includes withdrawing the delivery catheter and resected valve tissue from the vascular system of the patient. The method is preferably used to excise the aortic valve of an animal (including a human), but may alternatively be used to excise any cardiac valve or other valve in the cardiovascular system, or any suitable tissue accessible by catheter. The method may be used in conjunction with a percutaneous artificial valve replacement procedure.

Step S210, which includes the step of providing a system having proximal and distal frames, an electrode, and proximal and distal chambers, preferably includes providing the system for resecting a valve described above in Section 1, but may alternatively include providing elements of another suitable system deliverable by catheter. For example, instead of including an electrode for electrosurgical removal of the valve, the system may include a cutting blade that cuts through valve tissue.

Step S220, which includes the step of navigating a delivery catheter through the vascular system of a patient, preferably includes the steps of: introducing a central guidewire into the vascular system, navigating the central guidewire to the native valve, and introducing the delivery catheter and system over the guidewire. During step S220, the proximal and distal chambers are in the collapsed mode and sheathed within the delivery catheter. The central guidewire preferably is introduced into the vascular system by entering a femoral artery in a transfemoral approach, and navigated superiorly through the aorta, around the aortic arch, and towards the aortic valve. However, the central guidewire may alternatively be introduced into the vascular system through direct entry of the left ventricle of the heart in a transapical approach, or any suitable approach. The delivery catheter and system preferably are slipped over the guidewire and follow the path defined by the guidewire towards the aortic valve. The step of inserting the system into the vascular system of the patient is preferably similar that used in percutaneous aortic valve replacement. However, the guidewire, delivery catheter, and system may alternatively be inserted into the vascular system of the patient through any suitable process.

S230, which includes positioning the proximal and distal frames on opposite sides of the valve, preferably includes the steps of: passing the delivery catheter through the native valve until the distal frame is on the far side of the native valve, unsheathing the distal chamber and distal frame from the delivery catheter S234, allowing the distal chamber, distal frame, proximal chamber, and proximal frame to transform into their expanded modes S236, and actuating the proximal and distal frames towards each other such that the electrode is adjacent to the valve S238. The step S230 of positioning the proximal and distal frames on opposite sides of the valve preferably approaches the valve from one direction. Actuating the proximal and distal frames S238 preferably involves closing a gap defined by the proximal and distal frames in which the valve lies, which is preferably performed by turning an externally threaded drive cable coupled to the proximal and distal chambers. The gap may be a preset separation distance between the proximal and distal frames, such as a gap distance established during manufacturing and/or a gap distance established prior to operation by a surgeon, or any suitable gap distance. In one variation, one of the proximal chamber and distal chamber is fixed to the drive cable and the other chamber is held stationary, such that rotation of the drive cable rotates the distal chamber and moves the distal chamber towards and away from the proximal chamber, thereby opening and closing the gap between the distal and proximal frames. In a second variation, the exterior of the drive cable and either the proximal chamber or distal chamber include reverse (left-hand) threads, such that rotation of the drive cable causes the distal and proximal frames to move toward and away from one another, similar to a turnbuckle. During the step of actuating the proximal and distal frames, the proximal and distal frames and chambers are preferably operated in their expanded modes.

S240, which includes activating the electrode to resect the valve tissue, preferably includes providing electrocautery current to the electrode. Both the proximal and distal frames preferably include an electrode on opposing faces adjacent to the valve, such that the valve tissue that is positioned between the opposing distal and proximal frames is approached by the electrodes from two sides, thereby reducing required cutting time. The step of providing electrocautery current preferably involves the use of an external bipolar electrocautery or radiofrequency power supply connected to the distal and proximal frames through insulated current supply wires passing within the delivery catheter. Providing bipolar electrocautery current preferably cuts and excises the valve using electrosurgery technology. In other embodiments, the valve may additionally and/or alternatively be removed with include any radiofrequency or suitable form of energy, or with blade or other suitable cutting means.

S250, which includes capturing the resected valve tissue in the proximal and/or distal chambers, preferably involves filtering the blood surrounding the site of the valve with the use of mesh in the distal and/or proximal chambers. The mesh preferably allows passage of blood and other fluid, but contains resected valve tissue and/or other particles larger than the mesh size.

S260, which includes withdrawing the delivery catheter and resected valve tissue from the vascular system of the patient, preferably includes the steps of: returning the proximal and distal chambers to the collapsed mode S262, withdrawing the system into the delivery catheter, and removing the delivery catheter from the vascular system S264. The step S262 of returning the proximal and distal chambers to their collapsed modes preferably includes moving a compression ring S263 slidingly engaged with the distal chamber to radially compress the distal chamber into the collapsed mode. The compression ring is preferably located on the distal end of the distal chamber, which selectively slidingly engages with the distal chamber when pulled in a proximal direction with control wires, thereby causing the distal chamber to collapse and contract. The step of returning the proximal chamber and proximal frame to the collapsed mode preferably includes retracting the proximal chamber into the delivery catheter apex-first. During the step S264 of removing the delivery catheter from the vascular system of the patient, the proximal and distal frames and chambers are preferably operated in their collapsed modes. The steps of withdrawing the system into the delivery catheter and removing the delivery catheter from the vascular system are preferably similar to that used in percutaneous aortic valve replacement.

FIGS. 17A-17D illustrate another variation of an apparatus as described herein, highlighting another variation of a tissue engagement region. In FIG. 17A, the apparatus includes a distal capture chamber 1701 and a proximal capture region 1703, similar to those described above, which may include any of the elements or features described above (even if not explicitly shown in FIGS. 17A-17D), such as collapse actuators, etc. A drive cable or drive shaft 1705 passes through the midline of the apparatus, which is housed in a catheter 1721. A tissue engagement region 1707 is coupled to the drive wire 1705. FIGS. 17B and 17C show alternative views of the same apparatus. A transverse section through the view show in FIG. 17C (taken along plane 17D′) is shown in FIG. 17D.

FIG. 17D shows an enlarged view of the tissue engagement region. In this example, the tissue engagement region 1707 is formed of a plurality of toothed rings (e.g., rings having teeth). In this example, the teeth are angularly positioned around the ring, and oriented in the same direction (e.g., clockwise when looking distally down the apparatus). The teeth forming the tissue engagement region therefore project into the space between the distal and proximal chambers, forming a capture chamber or region between the distal and proximal chambers). Rotation of the drive cable clockwise will therefore rotate the teeth of the tissue engagement region within this space. This middle region between the distal and proximal chambers is positioned during use within the valve, and therefore the teeth of the tissue engagement region may engage (e.g., hook, snag, capture, etc.) the tissue of the valve, including the leaflets. As the tissue is cut by the apparatus, as described above, the tissue may be held by the tissue engagement region and even wrapped around the drive shaft centrally. Thus, when the distal and proximal chambers/frames are collapsed and withdrawn proximally, the tissue may be more easily retained. In apparatus in which the tissue capture region is attached to the central drive cable and moved dynamically, wrapping of the tissue around the drive shaft/cable may also help allow the proximal and distal chambers to collapse without interference.

Thus, FIGS. 17A-17C illustrate the transition of the drive cable in the middle region between the proximal and distal chambers into a toothed segmented region that is within the proximal and distal chambers. In this example, the toothed rings are shown as directional (e.g., all teeth point clockwise) but they may either be unidirectional or in different directions. For example, the teeth may be alternating in direction on subsequent rings, and/or alternating in direction around the perimeter of the rings.

The direction of the rings may provide a benefit. For example, alternating directional teeth may engage the tissue regardless of the direction the drive cable is rotated (clockwise or counterclockwise), while on-directional teeth may allow removal or loosening of the tissue engaged. As shown in FIGS. 17A-17D, the toothed segments may function in unison with the drive cable mechanism as they are directly connected, allowing a forward as well as a reverse longitudinal axis motion. In this example, the teeth forming the tissue engagement region may facilitate a grasping of the excised valve material, wrapping it about that segment of the drive cable and have the effect of drawing it inward to the cable and minimize the cross sectional area of the excised valve material and its subsequent removal through the guide catheter system, as mentioned above. The cable may utilize the forward or reverse mechanism of the drive cable system or a combination of both to accomplish this action.

Although the variations shown in FIGS. 17A-17D illustrate an apparatus in which the tissue engagement portion is moved with/by the drive shaft, in some variations the tissue engagement region may move independently of the drive shaft. For example, the tissue engagement region may be configured to rotate separately from the drive shaft. In some variations a separate drive shaft may be used.

In general, when a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements, these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. An apparatus for resecting a target cardiac valve in a patient, comprising:
a collapsible distal chamber having a proximally-facing opening;
a first annular cutter on the proximally-facing opening comprising a first plurality of nested and interconnectable frame segments;
a collapsible proximal chamber having a distally-facing opening;
a second annular cutter on the distally-facing opening comprising a second plurality of nested and interconnectable frame segments, wherein the first and second annular cutters are adapted to communicate to cut tissue of the target cardiac valve;
an elongate drive shaft extending distally to proximally through the distal chamber and the proximal chamber, wherein the elongate drive shaft is configured to reduce a proximal-to-distal separation between the distal chamber and the proximal chamber; and
a tissue engagement region within the distal chamber and the proximal chamber configured to engage and retain the tissue of the target cardiac valve within either or both the distal chamber and the proximal chambers;
wherein the distal and proximal chambers are configured to be collapsed around the drive shaft in a delivery configuration and further configured to expand for positioning on either side of the target cardiac valve.

2. The apparatus of claim 1, wherein the tissue engagement region is connected to the drive shaft and configured to rotate with the drive shaft.

3. The apparatus of claim 1, wherein the tissue engagement region comprises a mesh.

4. The apparatus of claim 1, wherein the tissue engagement region comprises a plurality of teeth.

5. The apparatus of claim 1, wherein the tissue engagement region comprises a plurality of rings coupled to the drive shaft, wherein the rings comprise teeth.

6. The apparatus of claim 1, wherein the tissue engagement region comprises a plurality of teeth coupled to the drive shaft intermediate between the distal chamber and the proximal chamber.

7. The apparatus of claim 1, wherein the first annular cutter comprises a first electrode and the second annular cutter comprises a second electrode, further wherein the first and second electrodes are configured to supply bipolar electrocautery current to electrosurgically resects the target valve tissue.

8. The apparatus of claim 1, wherein the proximal and distal chambers allows passage of fluid therethrough.

9. The apparatus of claim 1, further comprising a drive system that adjusts the distance between the proximal and distal rings.

10. The apparatus of claim 1, wherein the first plurality of nested and interconnectable frame segments have angled ends that complementarily mate with the angled ends of adjacent frame segments.

11. The apparatus of claim 1, wherein the first plurality of nested and interconnectable frame segments includes an arc segment having a radially inward angled end and another frame segment having a radially outward angled end that engages with the inward angled end when the distal chamber is expanded.

12. The apparatus of claim 1, further comprising a collapse actuator coupled to the distal chamber and configured to controllably collapse the distal chamber so that it may be withdrawn into a catheter.

13. The apparatus of claim 1, wherein the distal chamber includes a plurality of frame arms that collapse radially inwards towards the drive shaft when the chamber is collapsed and open radially outwards when the chamber is expanded.

14. The apparatus of claim 1, wherein at least one of the proximal and distal chambers is conical.

15. An apparatus for resecting a target cardiac valve in a patient, comprising:
a collapsible distal chamber having a proximally-facing opening;
a first annular cutter on the proximally-facing opening;
a collapsible proximal chamber having a distally-facing opening;
a second annular cutter on the distally-facing opening, wherein the first and second annular cutters are adapted to communicate to cut tissue of the target cardiac valve;
an elongate drive shaft extending distally to proximally through the distal chamber and the proximal chamber, wherein the elongate drive shaft is configured to reduce a proximal-to-distal separation between the distal chamber and the proximal chamber; and
a tissue engagement region comprising a plurality of teeth coupled to the drive shaft between the distal chamber and the proximal chamber and configured to dynamically engage and retain the target cardiac valve tissue within either or both the distal chamber and the proximal chambers;
wherein the distal and proximal chambers are configured to be collapsed around the drive shaft in a delivery configuration and further configured to expand for positioning on either side of the target cardiac valve.

16. The apparatus of claim 15, wherein the tissue engagement region is connected to rotate with the drive shaft.

17. The apparatus of claim 15, wherein the first annular cutter comprises a first electrode and the second annular cutter comprises a second electrode, further wherein the first and second electrodes are configured to supply bipolar electrocautery current to electrosurgically resects the target valve tissue.

18. The apparatus of claim 15, further comprising a collapse actuator coupled to the distal chamber and configured to controllably collapse the distal chamber so that it may be withdrawn into a catheter.

19. The apparatus of claim 15, wherein the distal chamber includes a plurality of frame arms that collapse radially inwards towards the drive shaft when the chamber is collapsed and open radially outwards when the chamber is expanded.

20. A method for minimally invasively resecting a cardiac valve, the method comprising:
positioning a catheter adjacent a target cardiac valve;
extending a distal chamber of an apparatus for resecting a target cardiac valve from the distal end of the catheter so that the distal chamber expands on a distal side of the cardiac valve and a proximally-facing opening of the distal chamber faces the cardiac valve;
extending a proximal chamber of the apparatus for resecting a target cardiac from the catheter so that the proximal chamber expands on a proximal side of the cardiac valve and a distally-facing opening of the proximal chamber faces the cardiac valve;
actuating a drive shaft of the apparatus to shorten the distance between the proximal and distal chambers;
engaging the valve tissue with a tissue engagement region on the drive shaft, wherein the tissue engagement region comprises a plurality of teeth;
cutting the valve tissue between a first annular cutter on the proximally-facing opening and a second annular cutter on the distally-facing opening to resect the valve tissue;
capturing the resected valve tissue in at least one of the proximal and distal chambers; and
withdrawing the delivery catheter and resected valve tissue from the vascular system of the patient.
